(19) United States
(12) Patent Application Publication
(10) Pub. No.: US 2015/0275404 A1
(43) Pub. Date: Oct. 1, 2015

(54) Nonwoven Having High Microbial Kill Rate And High Efficacy And Articles And Uses Therefrom

(71) Applicants: POLYMER GROUP, INC., Charlotee, NC (US); TRIOCIDE, INC., Plainfield, IL (US)

(72) Inventors: Sven Krister Erlandsson, Advance, NC (US); Frank M. Fosco, JR., Plainfield, IL (US); Ralph A. Moody, III, Mooresville, NC (US); Pierre D. Grondin, Mooresville, NC (US); John Frederick Steffen, Denver, NC (US)

(73) Assignees: POLYMER GROUP, INC., Charlotte, NC (US); TRIOCIDE, INC., Plainfield, IL (US)

(21) Appl. No.: 14/668,513

(22) Filed: Mar. 25, 2015

Related U.S. Application Data
(60) Provisional application No. 61/971,823, filed on Mar. 28, 2014.

(51) Int. Cl. D01F 8/04; A01N 33/12; A61L 31/14; A01N 33/04; D04H 1/4382; A01N 25/10; A01N 27/00

(52) U.S. Cl. CPC D01F 8/04; A01N 25/10; A01N 33/12; A01N 27/00; A01N 33/04; D04H 1/4382; A61L 31/14; D10B 2509/00; D10B 2401/13

(57) ABSTRACT

A fiber defined by a surface having a concentration of antimicrobial and a center having another concentration of antimicrobial is provided. The concentration of antimicrobial at the surface of the fiber is greater than the concentration of antimicrobial at the center of the fiber. Nonwovens manufactured from the fiber are also provided. The antimicrobial may include an antimicrobial heat labile component in conjunction with a carrier.

### CROSS-REFERENCE TO RELATED APPLICATIONS

This patent application claims the priority benefit of U.S. Provisional Application No. 61/971,823 filed on Mar. 28, 2014, the contents of which are incorporated herein by reference.

### FIELD OF INVENTION

The present invention relates to an antimicrobial nonwoven, articles manufactured therefrom, and uses for antimicrobial nonwovens of the present invention. The present invention also relates to the manufacture of an antimicrobial nonwoven.

### BACKGROUND

Conventional nonwovens having antimicrobial properties are known in the art. An exemplary use of such a nonwoven would be in a smock or scrub or gown worn by medical staff while working in the hospital. Advantageously, the product would have an efficacy and kill rate high enough to inactivate microbes in order to avoid cross contamination from patient to patient and patient to medical staff.

Nonwovens having antimicrobial properties conventionally include (1) an antimicrobial topical treatment applied to the nonwoven, (2) a metal-based antimicrobial that is added to the polymer used to form the fibers that constitute the nonwoven, and (3) an organic-based antimicrobial that is dispersed in to the polymer.

Nonwovens that include an antimicrobial topical treatment can demonstrate high efficacy and kill rate. However, the permanency of the effect is limited and limits the applicability of this type of antimicrobial nonwoven material. For example, an antimicrobial treatment applied topically to a nonwoven may be easily removed when the antimicrobial is contacted by a liquid or through abrasion on contact with some other object. Additionally, the antimicrobial may be subject to degradation upon exposure to heat perhaps through subsequent treatment of the nonwoven or converting the nonwoven for use as an article.

Fibers comprising metal-based antimicrobial additives, for example, silver nanoparticles, tend to have limited efficacy and kill rate, because only a fraction of the particles that are loaded in the polymer are available at the surface of the fiber. Additionally, these types of nonwovens have a higher cost due to the higher cost associated with the metal-based antimicrobial and the degree of loading needed to achieve a high enough surface concentration of the metal-base antimicrobial. The use of heavy metals, especially in disposable in disposable nonwovens, becomes less preferred.

Organic-based antimicrobials that are dispersed into a polymer of a fiber may be designed to bloom to the surface of the fiber thus overcoming the limitation of lower surface concentration associated with metal-based antimicrobial additives. For example, Triclosan that is dispersed into a polymeric formulation blooms to the surface as the polymer is extruded into a fiber. However, a limitation of these types of organic-based antimicrobials is the difficulty associated with retaining sufficient efficacy and kill rate due to the volatility of these types of compounds. Additionally, these compounds can become denatured upon being exposed to higher temperatures as the polymer is processed into fibers and further into a nonwoven material. While a higher concentration of these organic-based antimicrobials may be used to help offset these negative processing effects, there are limitations on the additional amounts that may be used. For example, increasing amounts of the organic-based antimicrobial may lead to an increase in drips of fiber breakage during the fiber spinning operation.

There remains a need for nonwovens and the articles made of nonwoven that exhibit high antimicrobial efficacy and high kill rate. There remains an unmet need for a nonwoven and articles manufactured therefrom having a high kill rate and a high efficiency that have an antimicrobial that can be included in a polymer used for the manufacture of fibers used in such a nonwoven that overcomes the disadvantages associated with conventional antimicrobial additives used in the manufacture of nonwoven materials.

### BRIEF SUMMARY

The present invention relates to a fiber defined by varying concentrations of antimicrobial throughout the cross section of the fiber. Without intending to be bound by theory, the fiber of the invention comprises an antimicrobial having an antimicrobial heat labile component in combination with a carrier. Yet other aspects of the invention relate to nonwovens manufactured from the fiber of the invention.

In one aspect, the invention provides a fiber, the fiber defined by a surface having a concentration of an antimicrobial and a center having another concentration of the antimicrobial. According to certain embodiments of the invention, the concentration of the antimicrobial at the surface of the fiber is greater than the concentration of the antimicrobial at the center of the fiber.

In an embodiment of the invention, the fiber has been constructed to have a surface area of at least about 1070 cm²/g.

In certain embodiments of the invention, the antimicrobial may comprise an antimicrobial heat labile component in combination with a carrier. In certain embodiments of the invention, the concentration of the antimicrobial at the surface of the fiber is from about 3.5 wt % to about 12 wt % based upon the total weight of the fiber. Further pursuant to this embodiment of the invention, the concentration of the antimicrobial at the center of the fiber may be less than about 50% of the concentration of the antimicrobial at the surface of the fiber.

In certain embodiments, the fiber of the invention is a bicomponent fiber defined by a sheath and a core, wherein the concentration of the antimicrobial in the sheath is greater than the concentration of the antimicrobial in the core. For example, according to certain embodiments of the invention, the concentration of the antimicrobial in the sheath may be from about 4 wt % to about 12 wt % based upon the total weight of the fiber. The concentration of the antimicrobial in the core is about 50% of the concentration of the antimicrobial in the sheath, according to certain embodiments of the invention.

In an embodiment of the invention, the kill rate of the fiber is at least about 95% (log₁₀) after 30 minutes as measured by AATCC 100 test according to certain embodiments of the invention or at least about 95% (log₁₀) after 3 minutes as measured by AATCC 100 test according to certain other embodiments of the invention.

Another aspect of the invention provides a nonwoven manufactured from a fiber having a surface concentration of an antimicrobial that is greater than a concentration of the antimicrobial at the center of the fiber.

Another aspect of the invention provides a method for manufacturing a fiber including the steps of dispersing an antimicrobial in a first polymer; and forming a sheath of the bicomponent fiber from the first polymer and a core of the bicomponent fiber from a second polymer.

According to certain embodiments, the method for manufacturing the fiber of the invention may additionally comprise the step of disposing the antimicrobial in the second polymer, wherein the concentration of the antimicrobial in the sheath is greater than the concentration of the antimicrobial in the core.

In certain embodiments of the invention, the antimicrobial of the method of manufacturing such a fiber may comprise an antimicrobial heat label component in combination with a carrier.

Other aspects and embodiments will become apparent upon review of the following description taken in conjunction with the accompanying drawing. The invention, though, is pointed out with particularity by the appended claims.

### BRIEF DESCRIPTION OF THE DRAWING

Having thus described the invention in general terms, reference will now be made to the accompanying drawing, and wherein:

FIG. 1 is a graphical representation of the percent bacterial reduction after three minutes compared to the percent loading of SMT 2000 masterbatch in the filament sheath.

### DETAILED DESCRIPTION OF THE INVENTION

The present invention now will be described more fully hereinafter, in which some, but not all embodiments of the invention necessarily being fully described. Preferred embodiments of the invention may be described, but this invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments of the invention are not to be interpreted in any way as limiting of the invention.

As used in the specification and in the appended claims, the singular forms “a”, “an”, and “the” include plural referents unless the context clearly indicates otherwise. For example, reference to “a fiber” includes a plurality of such fibers.

It will be understood that relative terms, such as “preceding” or “followed by” or the like, may be used herein to describe one element's relationship to another element. It will be understood that relative terms are intended to encompass different orders or orientations of the element. It will be understood that such terms can be used to describe the relative order or positions of the element or elements of the invention and are not intended, unless the context clearly indicates otherwise, to be limiting.

Although specific terms are employed herein, they are used in a generic and descriptive sense only and not for purposes of limitation. All terms, including technical and scientific terms, as used herein, have the same meaning as commonly understood by one of ordinary skill in the art to which this invention belongs unless a term has been otherwise defined. It will be further understood that terms, such as those defined in commonly used dictionaries, should be interpreted as having a meaning as commonly understood by a person having ordinary skill in the art to which this invention belongs. It will be further understood that terms, such as those defined in commonly used dictionaries, should be interpreted as having a meaning that is consistent with their meaning in the context of the relevant art and the present disclosure. Such commonly used terms will not be interpreted in an idealized or overly formal sense unless the disclosure herein expressly so defines otherwise.

The invention described herein relates to a nonwoven and any articles manufactured therefrom, where the nonwoven comprises one or several types of antimicrobial heat labile components absorbed on a solid carrier. In certain preferred embodiments of the invention, the nonwoven has an antimicrobial resistance that is measured as a kill efficiency of at least about 80% after 30 minutes, and, more preferably, a kill efficiency of at least about 90% after three (3) minutes.

The polymer dispersed heat labile antimicrobial technology used in the present invention is described more fully in U.S. Patent Application Publications No. 2013/0172436 entitled “Polymers Containing Heat Labile Components Adsorbed on Polymeric Carriers and Methods for Their Preparation” to Fosco, Jr et al.; 2014/0023690 entitled “Polymer Surfaces Containing Heat Labile Components Adsorbed on Polymeric Carriers” to Fosco, Jr. et al.; 2014/0023814 entitled “Potable Water Containers having Surfaces Including Heat Labile Component Carrier Combinations” to Fosco, Jr. et al.; and 2014/0011906 entitled “Surface Treatment Including a Heat Labile Component/Carrier Combination” to Fosco, Jr. et al. each of which are fully incorporated herein in their entirety by reference. The types of antimicrobial agents described in these publications were selected for use in the nonwovens of the invention because of their added stability offered by their adsorption on a carrier. In certain embodiments of the invention, the antimicrobials may be microencapsulated and absorbed on a carrier particle.

In an embodiment of this invention, a nonwoven web comprises staple fibers and has been stabilized by various methods including but not limited to thermal bonding, needling, or hydro-entangling. A nonwoven web may have been formed by any known method including but not limited to airlaid, wetlaid and carding process. In another embodiment of this invention the web may comprise fibers formed using a spunmelt process, including but not limited to a spunbond process and/or a meltblown process.

According to certain embodiments of the invention the nonwoven web comprises fibers or continuous filaments that are multi-components, the antimicrobial formulation being distributed in all the polymers forming the components, only in one of the polymers disposed toward the outside of the fibers, or less than all of the components of the multicomponent fiber in any arrangement. In an exemplary embodiment of the invention, the antimicrobial formulation may only be disposed in the sheath of a multicomponent fiber.

In another embodiment, the nonwoven of the invention may be used as a barrier fabric that is made from a combination of layers of continuous spunbond filaments and meltblown fibers. Both types of fiber may comprise the antimicrobial formulation or only one fiber may comprise the antimicrobial formulation. In a preferred embodiment of the invention, the antimicrobial formulation is contained only in the spunbond continuous filaments. In another embodiment of the invention, the continuous filaments are of the multi-component type and, the antimicrobial formulation is either essentially only disposed in or has a higher concentration in at least one polymer component located towards the outside of the filaments.

The terms “wt %” or “percent by weight” and the like should be construed as the wt % or percent by weight and the like calculated based upon the total weight of the object (e.g., fiber, nonwoven, etc.) in question unless the context in which it is disclosed clearly describes otherwise.

As used herein, the term “antimicrobial” means one or more agents, materials, heat labile components, and any combination thereof, or a surface containing the one or more agents, materials, heat labile components, and any combination thereof that will kill, inhibit the growth of, control, or prevent the formation of microbes from any one or more of the families consisting of bacteria, viruses, and fungi. Examples of such microbes may include, but are not limited to Aureobasidium pullulans, Bacillus cereus, Bacillus thuringiensis, Chaetomium globosum, Enterobacter aerogines, Escherichia coli, Gliocladtum Wrens, Klebsiella Pheumoniae, Legionella pneumpophila, Listeria Monocytogenes, Mycobacterium tuberculosis, Porphyromonas girrgivalis, Proteus mirabilis, Proteus vulgaris, Pseudomonas aeruginosa, Saccharomyces cerevisiae, Salmonella gallinarum, Salmonella typhimurium, Staphylococcus aureus, Staphylococcus epidermidis, Streptococcus agalactiae, Streptococcus faecalis, Streptococcus mutans, Trycophyton malmsten, Vibrio parahaemolyticus, Stachybotrys, Aspergillus niger, Candida albicans and Penicillium fimiculosum.

An aspect of the invention provides an article comprising the nonwoven of the invention. An exemplary embodiment of such an article is an article that is used in environment where it is desirable to avoid microbial cross contamination. For example, non-limiting examples of articles of the invention, include an outer garment comprising the nonwoven of the invention like, for example, a smock, a scrub, a lab coat, a shoe cover, a gown, a cap, a face mask, a protective apparel, or a sleeve protector. Other non-limiting examples of articles comprising the nonwoven of the invention are a drape, any type of linen for bedding as an example, a separation screen, a fabric used in agricultural application, a filter, and a wipe.

The inventors have discovered that by including at least one type of antimicrobial heat labile component on a carrier in the fibers used in the nonwovens of the invention, a high kill rate and a high efficacy is achieved. Without intending to be bound by the theory, the exceptional performance improvement may be due to the combination of the proper choice of antimicrobial heat labile component used and a high surface to weight ratio that can be achieved in the fibers of the nonwoven, the latter resulting in a reduction in the distance the antimicrobial heat labile component absorbed on the carrier must travel to bloom to and reach the surface of the fiber.

In an embodiment, the nonwoven of the invention comprises fibers containing an antimicrobial composition, the fibers of the nonwoven being less than about 10 decitex in size; preferably, less than about 5 decitex; and, more preferably, less than about 3.5 decitex. In certain embodiments of the invention, the nonwoven comprises a mixture of fibers where the fibers comprising the antimicrobial are less than about 10 decitex in size; preferably, less than about 5 decitex; and, more preferably, less than about 3.5 decitex. According to certain embodiments of the invention, the nonwoven comprises more than one type of fiber where the fiber having the antimicrobial composition is at least about 10 wt %, at least about 20 wt %, at least about 30 wt %, at least about 40 wt %, at least about 50 wt %, at least about 60 wt %, at least about 70 wt %, at least about 80 wt %, at least about 90 wt %, at least about 95 wt %, or at least about 98 wt % based upon the total weight of the nonwoven.

In certain embodiments of the invention, the fiber comprising the antimicrobial is constructed to maximize surface area per unit weight of the fiber. Without intending to be bound by theory, a fiber having a large surface area and an antimicrobial selected such that it preferably is concentrated at the surface of the fiber can maximize kill rate per unit antimicrobial used in the fiber. For example, an antimicrobial that comprises an antimicrobial heat labile component in combination with a carrier that migrates or blooms to the surface during processing of the fiber and/or the associated nonwoven may be preferred, according to certain embodiments of the invention.

According to an embodiment of the invention, the fiber comprising the antimicrobial may have a surface area of at least about 1070 cm²/g; preferably at least about 1506 cm²/g; and, more preferably, at least about 1814 cm²/g. In an embodiment of the invention, the antimicrobial heat labile component and carrier combination are selected such the concentration of the antimicrobial is greater towards the outside surface of the fibers of the nonwoven of the invention. In another embodiment of the invention, the fiber may be a multicomponent fiber, such as a bicomponent fiber, where the antimicrobial heat labile component and carrier are disposed in the sheath of the fiber. More preferably, upon forming the fiber, the antimicrobial component becomes more concentrated towards the outer surface of the sheath. Without intending to be bound by theory, concentrating the antimicrobial towards the outer surface of the fiber enables the nonwoven manufactured from such a fiber to have a greater kill rate.

The antimicrobial heat labile component and carrier may be part of a masterbatch that is combined with the polymer of the fiber. Without intending to be bound by theory, the addition of the masterbatch may help stabilize the fiber spinning process and allow for very high loadings of the masterbatch to be achieved effectively providing increased antimicrobial efficacy and a fast kill rate to be achieved. In certain embodiments of the invention, the fiber comprises about 50 wt % or less; about 40 wt % or less; about 25 wt % or less; or about 15 wt % or less of the masterbatch based on the total weight of the fiber.

In addition to the antimicrobial heat labile component, a masterbatch may comprise a single polymer as described herein or a polymer blend based upon any of the combinations as described herein. The weight ratio of the single polymer or polymer blend to the antimicrobial heat labile component in the masterbatch may be from about 10:1 to about 1:4, preferably about 3:2. According to certain embodiments of the invention, the masterbatch is concentrated relative to the antimicrobial heat labile component. This masterbatch may have a weight ratio of the single polymer or polymer blend to the antimicrobial heat labile component in the masterbatch may be from about 4:1 to about 1:4, from about 3:1 to about 1:3, or from about 2:1 to about 1:2, or about 1.25:1.

According to certain embodiments of the invention, the masterbatch may be less concentrated relative to the antimicrobial heat labile component. This concentrated masterbatch may have a weight ratio of the single polymer or polymer blend to the antimicrobial heat labile component in the masterbatch may be from 4:1 to about 1:4, from about 3:1 to about 1:3, or from about 2:1 to about 1:2, or about 1.25:1.

In certain embodiments of the invention, the masterbatch may additionally comprise other additives. For example, according to an embodiment of the invention, the masterbatch may comprise an additive to better control the viscosity of the masterbatch. In certain preferred embodiments of the invention, one or more additives in the masterbatch combined with a polymer allow the extrusion temperature of the combined masterbatch polymer material to be lowered relative to a combination substantially free of any such additives. According to an embodiment of the invention, the masterbatch includes a silicone. In certain embodiments of the invention, the masterbatch includes a high molecular weight silicone. In certain embodiments of the invention, the high molecular weight silicone has a concentration of at least about 1 wt %, at least about 2 wt %, at least about 4 wt %, at least about 5 wt %, at least about 7 wt %, at least about 10 wt %, at least about 12 wt %, at least about 15 wt %, or at least about 20 wt % based on the total weight of the masterbatch. In yet other embodiments of the invention, the high molecular weight silicone may be added to the polymer separate from the masterbatch, yet in the same proportions relative to the masterbatch as already disclosed herein.

According to an embodiment of the invention, the antimicrobial heat labile component may comprise any of didecyldimethyl ammonium chloride, quaternary ammonium compounds, benzyl C-12-16 alkylidimethyl chlorides, benzathonium chloride, cetrimonium chloride, N-(3-aminopropyl)-N-dodecylpropane-1,3 diamine, and any combination thereof. In certain embodiments of the invention, the antimicrobial heat labile component may comprise from about 0 wt % to about 30 wt % of didecyldimethyl ammonium chloride, from about 0 wt % to about 22 wt % of quaternary ammonium compounds, from about 0 wt % to about 22 wt % of benzyl C-12-16 alkylidimethyl chlorides, from about 1 wt % to about 22 wt % of benzathonium chloride, from about 1 wt % to about 22 wt % of cetritnonium chloride, and from about 1 wt % to about 22 wt % of N-(3-aminopropyl)-N-dodecylproparte-1,3 diamine. According to a specific embedment of the invention, the antimicrobial heat labile component may comprise about 30 wt % of didecyldimethyl ammonium chloride, 22 wt % of benzyl C-12-16 alkylidimethyl chlorides, 17 wt % of benzathonium chloride, 9 wt % of cetrimonium chloride, 22 wt % of N-(3-aminopropyl)-N-dodecylpropane-1,3 diamine. According to another specific embodiment of the invention, the antimicrobial heat labile component may comprise about 30 wt % of didecyldimethyl ammonium chloride, 22 wt % of quaternary ammonium compounds, 17 wt % of benzathonium chloride, 9 wt % of cetrimonium chloride, 22 wt % of N-(3-aminopropyl)-N-dodecylpropane-1,3 diamine.

In an embodiment, a polymer of a fiber or even a combination of polymers in a multicomponent fiber for use in the nonwoven of the invention, are selected such that the migration of the antimicrobial component towards the center (or the core in the case of a multi-component fiber) is minimized. In certain embodiments of the invention, the fiber is a multi-component fiber having a core comprising a first polymer surrounding by at least one sheath comprising a second polymer where the antimicrobial heat labile component has a lower solubility in the first polymer in comparison to the solubility of the antimicrobial heat labile component in the second polymer.

In an embodiment of the invention, the concentration of an antimicrobial at the surface of a fiber is greater than a concentration of the antimicrobial at the center of the fiber. In certain embodiments of the invention, this preferred distribution of the antimicrobial may be achieved through proper selection of an antimicrobial heat labile component and carrier combination according to the teachings provided herein. Further pursuant to this embodiment, the preferred distribution may be achieved through proper selection of the polymer or polymers used in the fiber. In certain other embodiments of the invention, this preferred distribution may be achieved with the use of a multicomponent fiber where the concentration of the antimicrobial in the outer sheath of the multicomponent fiber is greater than the concentration of the antimicrobial in the core of the multicomponent fiber. In a preferred embodiment of the invention, the fiber is a bicomponent fiber and the concentration of antimicrobial in the sheath of the bicomponent fiber is greater than the concentration of antimicrobial in the core of the bicomponent fiber.

According to certain embodiments of the invention, the concentration of antimicrobial in the fiber may be from about 0.1 wt % to about 50 wt % based upon the total weight of the fiber. In certain embodiments of the invention, the concentration of antimicrobial in the fiber may be from about 0.25 wt % to about 30 wt % based upon the total weight of the fiber. In certain other embodiments of the invention, the concentration of antimicrobial in the fiber may be from about 0.5 wt % to about 25 wt % based upon the total weight of the fiber. In yet certain other embodiments of the invention, the concentration of antimicrobial in the fiber may be from about 1 wt % to about 20 wt % based upon the total weight of the fiber. In still yet certain other embodiments of the invention, the concentration of antimicrobial in the fiber may be from about 1 wt % to about 10 wt % based upon the total weight of the fiber. In even yet other embodiments of the invention, the concentration of antimicrobial in the fiber may be from about 0.25 wt % to about 5 wt %, from about 0.25 wt % to about 2.5 wt %, or from about 6 wt % to about 20 wt % based upon the total weight of the fiber.

In certain preferred embodiments of the invention, the antimicrobial will have a concentration distribution gradient within the fiber where, on average, a high concentration of antimicrobial will be found at the surface of the fiber and, on average, a lower concentration of antimicrobial relative to the concentration of antimicrobial at the surface of the fiber will be found at about the center of the fiber.

According to certain embodiments of the invention, the concentration of antimicrobial at the surface of the fiber may be from about 0.1 wt % to about 50 wt % based upon the total weight of the fiber. In certain embodiments of the invention, the concentration of antimicrobial at the surface of the fiber may be from about 0.5 wt % to about 40 wt % based upon the total weight of the fiber. In certain other embodiments of the invention, the concentration of antimicrobial at the surface of the fiber may be from about 1 wt % to about 30 wt % based upon the total weight of the fiber. In yet certain other embodiments of the invention, the concentration of antimicrobial at the surface of the fiber may be from about 1 wt % to about 25 wt % or from about 1 wt % to about 40 wt % based upon the total weight of the fiber. In still yet certain other embodiments of the invention, the concentration of antimicrobial at the surface of the fiber may be from about 2 wt % to about 25 wt % based upon the total weight of the fiber. In even yet other embodiments of the invention, the concentration of antimicrobial at the surface of the fiber may be from about 5 wt % to about 25 wt % or from about 6 wt % to about 20 wt % based upon the total weight of the fiber. In even still yet other embodiments of the invention, the concentration of antimicrobial at the surface of the fiber may be from about 3.5 wt % to about 12 wt % or from about 4 wt % to about 10 wt % based upon the total weight of the fiber.

According to certain embodiments of the invention, the concentration of antimicrobial at the center of the fiber is at most about 50%, at most about 40%, at most about 30%, at most about 25%, at most about 20%, at most about 15%, at most about 10%, at most about 5%, at most about 2%, or at most about 1% of the concentration of antimicrobial at the surface of the fiber. In certain embodiments of the invention, there is substantially no antimicrobial at the center of the fiber.

According to an embodiment of the invention, the fiber is a multicomponent fiber having at least one sheath and at least one core. For example, in certain embodiments of the invention, the antimicrobial fiber is a bicomponent fiber having a sheath and a core. Further pursuant to these embodiments, the concentration of antimicrobial in the sheath of the multicomponent fiber may be from about 0.1 wt % to about 50 wt % based upon the total weight of the sheath of the fiber. In certain embodiments of the invention, the concentration of antimicrobial in the sheath of the multicomponent fiber may be from about 0.25 wt % to about 30 wt % based upon the total weight of the sheath of the fiber. In certain other embodiments of the invention, the concentration of antimicrobial in the sheath of the multicomponent fiber may be from about 0.5 wt % to about 25 wt % based upon the total weight of the sheath of the fiber. In yet certain other embodiments of the invention, the concentration of antimicrobial in the sheath of the multicomponent fiber may be from about 1 wt % to about 20 wt % based upon the total weight of the sheath of the fiber. In still yet certain other embodiments of the invention, the concentration of antimicrobial in the sheath of the multicomponent fiber may be from about 1 wt % to about 10 wt % based upon the total weight of the sheath of the fiber. In even yet other embodiments of the invention, the concentration of antimicrobial in the sheath of the multicomponent fiber may be from about 0.25 wt % to about 5 wt %, from about 0.25 wt % to about 2.5 wt %, or from about 6 wt % to about 20 wt % based upon the total weight of the sheath of the fiber.

According to certain embodiments of the invention, the concentration of antimicrobial at the surface of the sheath of the multicomponent fiber may be from about 0.1 wt % to about 50 wt % based upon the total weight of the sheath of the fiber. In certain embodiments of the invention, the concentration of antimicrobial at the surface of the sheath of the multicomponent fiber may be from about 0.5 wt % to about 40 wt % based upon the total weight of the sheath of the fiber. In certain other embodiments of the invention, the concentration of antimicrobial at the surface of the sheath of the multicomponent fiber may be from about 1 wt % to about 30 wt % based upon the total weight of the sheath of the fiber. In yet certain other embodiments of the invention, the concentration of antimicrobial at the surface of the sheath of the multicomponent fiber may be from about 1 wt % to about 25 wt % or from about 1 wt % to about 40 wt % based upon the total weight of the sheath of the fiber. In still yet certain other embodiments of the invention, the concentration of antimicrobial at the surface of the sheath of the multicomponent fiber may be from about 2 wt % to about 25 wt % based upon the total weight of the sheath of the fiber. In even yet other embodiments of the invention, the concentration of antimicrobial at the surface of the sheath of the multicomponent fiber may be from about 5 wt % to about 25 wt % or from about 6 wt % to about 20 wt % based upon the total weight of the sheath of the fiber.

According to certain embodiments of the invention, the concentration of antimicrobial at the core of the multicomponent fiber is at most about 50%, at most about 40%, at most about 30%, at most about 25%, at most about 20%, at most about 15%, at most about 10%, at most about 5%, at most about 2%, or at most about 1% of the concentration of antimicrobial at the surface of the sheath of the multicomponent fiber. In certain embodiments of the invention, there is substantially no antimicrobial at the core of the multicomponent fiber.

According to another embodiment of the invention, the polymer comprising the antimicrobial heat labile component may additionally comprise at least one component selected for its ability to accelerate the blooming or migration of the antimicrobial heat labile component to the surface of the fiber. The selection of the carrier may also control the ability of the antimicrobial heat label component/carrier combination to properly migrate to the surface of the fiber.

Either in addition to or as an alternative to the ability of the antimicrobial to bloom or migrate to the surface of the fiber, at least two masterbatches may be used in the formation of the fiber. One of the at least two masterbatches will have a higher concentration of antimicrobial and, preferably, will be used in the formation of the outer portion of the fiber or, in the case of a multicomponent or bicomponent fiber, will be used in the formation of the sheath of the multicomponent or bicomponent fiber. Another of the at least two masterbatches will have a lower concentration of antimicrobial relative to the concentration of the aforementioned high concentration masterbatch, and, preferably, will be used in the formation of the inner portion of the fiber or, in the case of a multicomponent or bicomponent fiber, will be used in the formation of the core of the multicomponent or bicomponent fiber.

In another embodiment of the invention, a masterbatch having a higher concentration of antimicrobial will be used in the formation of the inner portion of the fiber or, in the case of a multicomponent or bicomponent fiber, will be used in the formation of the core of the multicomponent or bicomponent fiber, and a masterbatch having a lower concentration of antimicrobial relative to the concentration of antimicrobial in the aforementioned masterbatch will be used in the formation of the outer portion of the fiber or, in the case of a multicomponent or bicomponent fiber, will be used in the formation of the sheath of the multicomponent or bicomponent fiber.

The polymer of the polymer/antimicrobial heat labile component of the invention may be a thermoplastic polymer or a blend of a combination of thermoplastic polymers. According to an embodiment of the invention, the polymer may be a polyolefin including one or a combination of polyethylene and polypropylene. In another embodiment of the invention at least about 50 wt % of the polymer comprises a polyolefin including one or a combination of polyethylene and polypropylene. Polyethylene and polypropylene are used here in the broadest sense to include homopolymer, copolymers and funtionalized versions of these polymers. Polypropylene may also include the various forms of tacticity including isotactic, syndiotactic, atactic, and any combination of these types of tacticity. In an embodiment of the invention, the polypropylene may be manufactured by using a Ziegler-Natta or a metallocene catalyst.

The fibers used in the manufacture of the nonwoven of the invention may include one or several antimicrobial heat labile components absorbed on a carrier, where this combination is substantially unmodified at the temperatures used to form the fibers of the invention and subsequent processing of the nonwoven comprising such fibers.

The nonwoven of the invention comprises at least one fiber containing the antimicrobial heat label component that is dispersed in a thermoplastic polymer or blend of thermoplastic polymers. Thermoplastic polymers may include polymers that can be made to flow and processed into fibers upon being heated. Examples of thermoplastic polymers include, but are not limited to polyolefins, polyesters, polyamides, copolyamide, fluoropolymer, polyvinyl alcohol, polyvinyl acetate, polyethylene oxide, and polyacetal. In certain preferred embodiments of the invention, the polymer comprises a polyolefin or a blend of polyolefin polymers. The polyolefin polymers may be manufactured using certain synthesis approaches including, for example, catalyst systems commonly known as Ziegler-Nata, metal Eocene, or single site catalysts (SSC).

In certain embodiments of the invention, the polyolefin may comprise any one or combination of polypropylene, polyisobutylene, polybut-1-ene, poly-4-methylpent-1-ene, polyisoprene or polybutadiene, as well as polymers of cycloolefins, for instance of cyclopentene or norbornene, polyethylene, as well as copolymers comprising ethylene or propylene as main building block. Examples for those are without being limited to, copolymers of monoolefins and diolefins with each other or with other vinyl monomers, for example ethylene/propylene copolymers, linear low density polyethylene (LLDPE) and mixtures thereof with low density polyethylene (LDPE), propylene/but-1-ene copolymers, propylene/isobutylene copolymers, ethylene/but-1-ene copolymers, ethylene/hexene copolymers, ethylene/methylpentene copolymers, ethylene/heptene copolymers, ethylene/octene copolymers, propylene/butadiene copolymers, isobutylene/isoprene copolymers, ethylene/alkyl acrylate copolymers, ethylene/alkyl methacrylate copolymers, ethylene/vinyl acetate copolymers and their copolymers with carbon monoxide or, ethylene/acrylic acid copolymers and their salts (ionomers) as well as terpolymers of ethylene with propylene and a diene such as hexadiene-dicyclopentadiene or ethylidene-norbornene; and mixtures of such copolymers with one another and with polymers mentioned in 1) above, for example polypropylene/ethylene-propylene copolymers, LDPE/ethylene-vinyl acetate copolymers (EVA), LDPE/ethylene-acrylic acid copolymers (EM), LLDPE/EVA, and LLDPE/EM.

In certain embodiments of the invention, the polymer may be a mixture of polymers comprising a major component that is a polyolefin polymer. For example, these mixture of polymers may comprise polypropylene with polyisobutylene, polypropylene with polyethylene (for example PP/HDPE, PP/LDPE), or mixtures of different types of polyethylene (for example LDPE/HDPE).

According to an embodiment of the invention, the temperature at which the polymer and antimicrobial combination is extruded is minimized to help prevent degradation of the antimicrobial. In an embodiment of the invention, the temperature at which the polymer and antimicrobial combination is extruded is not more than about 15° C., not more than about 20° C., not more than about 25° C., not more than about 30° C., not more than about 35° C., not more than about 40° C., not more than about 45° C., or not more than about 50° C. over the melting temperature of the polymer and antimicrobial combination.

The antimicrobial heat labile component of the invention is adsorbed on a carrier, for example, a carrier particle according to an embodiment of the invention. The antimicrobial heat labile component alone and unassociated with the carrier would not be capable of withstanding the processing conditions required to reduce the polymer of the fiber to a molten state required in forming the fiber.

Antimicrobial compounds or biocides utilized according to the present disclosure are generally biocides which have reduced stability when exposed to required processing conditions at temperatures above their decomposition or volatilization temperature. Many biocides have limited stability upon being heated that prevent their incorporation into polymers using conventional methods.

Biocides generally suitable for processing according to the current disclosure in combination with a carrier include, but are not limited to: Acetylcarnitine, Acetylcholine, Aclidinium bromide, Acriflavinium chloride, Agelasine, Aliquat 336, Ambenonium chloride, Ambutonium bromide, Aminosteroid, Anilinium chloride, Atracurium besilate, Benzalkonium chloride, Benzethonium chloride, Benzilone, Benzododeciniurn bromide, Benzoxonium chloride, Benzyltrimethylammonium fluoride, Benzyltrimethylammonium hydroxide, Bephenium hydroxynaphthoate, Berberine, Betaine, Bethanechol, Bevonium, Bibenzonium bromide, Bretylium, Bretylium for the treatment of ventricular fibrillation, Burgess reagent, Butylscopolamine, Butyrylcholine, Candocuronium iodide, Carbachol, Carbethopendecinium bromide, Carnitine, Cefluprenam, Cetrimonium, Cetrimonium bromide, Cetrimonium chloride, Cetylpyridinium chloride, Chelerythrine, Chlorisondamine, Choline, Choline chloride, Cimetropium bromide, Cisatracurium besilate, Citicoline, Clidinium bromide, Clofilium, Cocamidopropyl betaine, Cocamidopropyl hydroxysultaine, Complanine, Cyanine, Decamethonium, 3-Dehydrocarnitine, Demecarium bromide, Denatonium, Dequalinium, Didecyldimethylammonium chloride, Dimethyldioctadecylammonium chloride, Dimethylphenylpiperazinium, Dimethyltubocurarinium chloride, DiOC6, Diphemanil metilsulfate, Diphthamide, Diquat, Distigmine, Domiphen bromide, Doxacurium chloride, Echothiophate, Edelfosine, Edrophonium, Emepronium bromide, Ethidium bromide, Euflavine, Fenpiverinium, Fentonium, Gallamine triethiodide, Gantacuriurn chloride, Glycine betaine aldehyde, Glycopyrrolate, Guar hydroxypropyltrimonium chloride, Hemicholinium-3, Hexafluoronium bromide, Hexamethonium, Hexocyclium, Homatropine, Hydroxyethylpromethazine, Ipratropium bromide, Isometamidium chloride, Isopropamide, Jatrorrhizine, Laudexiuin metilsulfate, Lucigenin, Mepenzolate, Methacholine, Methantheline, Methiodide, Methscopolamine, Methylatropine, Methylscopolamine, Metocurine, Miltefosine, MPP+, Muscarine, Neurine, Obidoxime, Otilonium bromide, Oxapium iodide, Oxyphenonium bromide, Palmatine, Pancuronium bromide, Pararosaniline, Pentamine, Penthienate, Pentolinium, Perifosine, Phellodendrine, Phosphocholine, Pinaverium, Pipecuronium bromide, Pipenzolate, Poldine, Polyquaternium, Pralidoxime, Prifinium bromide, Propantheline bromide, Prospidium chloride, Pyridostigmine, Pyrvinium, Quaternium-15, Quinapyramine, Rapacuronium, Rhodamine B, Rocuronium bromide, Safranin, Sanguinarine, Stearalkonium chloride, Succinylmonocholine, Suxamethonium chloride, Tetra-n-butylamtnonium bromide, Tetra-n-butylammonium fluoride, Tetrabutylammonium hydroxide, Tetrabutylammonium tribromide, Tetraethylammonium, Tetraethylammonitum bromide, Tetramethylammonium chloride, Tetramethylammonium hydroxide, Tetratnethylammonium pentafluoroxenate, Tetrabutylammonium bromide, Tetrapropylammonium perruthenate, Thiazinamium metilsulfate, Thioflavin, Thonzonium bromide, Tibezonium iodide, Tiemonium iodide, Timepidium bromide, Trazium, Tridihexethyl, Triethylcholine, Trigonelline, Trimethyl ammonium compounds, Trimethylglycine, Trolamine salicylate, Trospium chloride, Tubocurarine chloride, and Vecuronium bromide.

Preferred antimicrobial heat labile compounds include, but are not limited to, quaternary amines and antibiotics. Some specific preferred antimicrobial heat labile compounds include, but are not limited to, N,N-didecyl-N-methyl-N-(3-trimethoxysilylpropyl)ammonium chloride, cetyl pyridinium chloride, N,N-bis(3-aminopropyl)dodecylamine, N-octyl-N-decyl-N-dimethyl-ammonium chloride, N-di-octadecyl-N-dimethyl-ammonium chloride, and N-didecyl-N-dimethyl-ammonium chloride.

Antibiotics may include, but are not limited to, amoxicillin, campicillin, piperacillin, carbenicillin indanyl, methacillin cephalosporin cefaclor, streptomycin, tetracycline and the like. Preferred combinations of biocides generally include at least one antimicrobial heat labile component, which would not survive incorporation into a specific polymer unless adsorbed onto a carrier.

Suitable carriers of the invention are typically porous materials capable of adsorbing the antimicrobial heat labile component, remaining in a solid form without decomposition during processing of a polymer in a molten phase, and maintaining the antimicrobial in the adsorbed state during processing. Carriers having a substantial porosity and a high surface area (mostly internal) are suitable. A further useful property for a carrier is a relatively low thermal conductivity. Finally, for some applications, carriers that do not alter the color or appearance of the polymer are particularly suitable.

Carriers that can be used in the invention include, but are not limited to, inorganics such as platy minerals and polymers. Examples of inorganics include, but are not limited to fumed and other forms of silicon including precipitated silicon and vapor deposited silicon; clay; kaolin; perlite bentonite; talc; mica; calcium carbonate; titanium dioxide; zinc oxide; iron oxide; silicon dioxide; and the like. Mixtures of a combination of carriers may also be used. Polymeric carriers should remain solid at elevated temperatures and be capable of loading sufficient quantities of antimicrobial either into a pore system or through other means of incorporation. Suitable polymeric carriers may include, but are not limited to, organic polymeric carriers such as cross-linked macroreticular and gel resins, and combinations thereof such as the so-called plum pudding polymers. Additional carriers suitable for use in certain embodiments of the invention include organic polymeric carriers such as porous macroreticular resins, some of which may include other resins within the polymer's structure. Suitable resins for imbedding within a macroreticular resin include other macroreticular resins or gel resins. Additionally, other porous non-polymeric materials such as minerals can similarly be incorporated within the macroreticular resin according to certain embodiments of the invention.

Organic polymeric carriers suitable for certain embodiments of the invention may include polymers lacking a functional group, such as a polystyrene resin, or carriers having a functional group such as a sulfonic acid included. Generally, any added functional group should not substantially reduce the organic polymeric carrier's thermal stability. A suitable organic polymeric carrier should be able to load a sufficient amount of biocide, and survive any processing conditions, and deliver an effective amount of the heat labile component such as a biocide upon incorporation into any subsequent system. Suitable organic polymeric carriers can be derived from a single monomer or a combination of monomers. Combinations of inorganic and organic carriers can be utilized.

Any general method for preparing macroreticular and gel polymers that is well known in the art utilizing a variety of monomers and monomer combinations may be used. Suitable monomers for the preparation of organic polymeric carriers include, but are not limited to styrene, vinyl pyridines, ethylvinylbenzenes, vinyltoluenes, vinyl imidazoles, an ethylenically unsaturated monomers, such as, for example, acrylic ester monomers including methyl acrylate, ethyl acrylate, butyl acrylate, 2-ethylhexyl acrylate, decyl acrylate, methyl methacrylate, butyl methacrylate, lauryl (meth)acrylate, isobornyl (meth)acrylate, isodecyl (meth)acrylate, oleyl (meth)acrylate, palmityl (meth)acrylate, stearyl (meth)acrylate, hydroxyethyl (meth)acrylate, and hydroxypropyl (meth)acrylate; acrylamide or substituted acryl amides; styrene or substituted styrenes; butadiene; ethylene; vinyl acetate or other vinyl esters such as vinyl acetate, vinyl propionate, vinyl butyrate and vinyl laurate; vinyl ketones, including vinyl methyl ketone, vinyl ethyl ketone, vinyl isopropyl ketone, and methyl isopropenyl ketone; vinyl ethers, including vinyl methyl ether, vinyl ethyl ether, vinyl propyl ether, and vinyl isobutyl ether; vinyl monomers, such as, for example, vinyl chloride, vinylidene chloride, N-vinyl pyrrolidone; amino monomers, such as, for example, N,N′-dimethylamino (meth)acrylate; and acrylonitrile or methacrylonitrile; and the monomethacrylates of dialkylene glycols and polyalkylene glycols. Descriptions for making porous and macroreticular polymers can be found in U.S. Pat. No. 7,422,879 to Gebhard et al. and U.S. Pat. No. 7,098,252 to Jiang et al.

Organic polymeric carriers may contain other organic polymeric particles and/or other inorganic carrier particles, such as minerals typically characterized as platy materials. Minerals suitable for incorporation into a polymeric carrier include, but are not limited to fumed and other forms of silicon including precipitated silicon and vapor deposited silicon; clay; kaolin; perlite bentonite; talc; mica; calcium carbonate; titanium dioxide; zinc oxide; iron oxide; silicon dioxide; and the like. Mixtures of different carriers may also be utilized according to certain embodiments of the invention.

Nonwovens of the invention comprise fiber and filaments containing an antimicrobial formulation of the invention where such antimicrobial is available at the surface of the fiber. The nonwoven of the invention may comprise only fibers of the invention having antimicrobial or a combination of fibers of the invention and other fibers that may include conventional antimicrobial additives and/or substantially free of any antimicrobial.

The fibers and/or filaments of the invention, used in the manufacture of nonwovens of the inventions will result in an improved kill rate and efficacy over fibers and nonwovens currently known in the art. In certain embodiments of the invention, the AATCC 100 bacterial reduction after 3 minutes in the log₁₀ values is at least about 20%, at least about 30%, at least about 34%, at least about 40%, at least about 50%, at least about 60%, at least about 70%, at least about 80%, or at least about 90%. According to certain preferred embodiments of the invention, the AATCC 100 bacterial reduction after 3 minutes in the logᵢₙ values is at least about 90%, at least about 95%, at least about 96%, at least about 97%, at least about 98%, at least about 99%, or at least about 99.9%. According to certain other embodiments of the invention, the AATCC 100 bacterial reduction after about 30 minutes in the log₁₀ values is at least about 90%, at least about 95%, at least about 96%, at least about 97%, at least about 98%, at least about 99%, or at least about 99.9%.

The nonwovens of the invention may be manufactured from any method suitable for formulations comprising thermoplastic fibers. The nonwovens may comprise staple fibers containing the antimicrobial formulation according to the teachings provided herein, where such staple fibers may be as short as 3 mm or up to as long as 150 mm. The staple fibers may be crimped or not crimped or a combination of crimped fibers and fibers that are not crimped. The nonwovens of the invention may also comprise substantially continuous filaments, the filaments containing the antimicrobial formulation of the invention. The filaments may also be crimped or not crimped or a combination of crimped filaments and filaments that have not been crimped. The staple fibers or continuous filaments of the invention may have a homogeneous composition or can be composed of a multicomponent type including, but not limited to, sheath/core bicomponent, core/sheath/sheath bicomponent or tri-component, and side-by-side bicomponent. A preferred structure of a multi-component staple fiber or filament is the sheath/core construction where the sheath comprises from about 5 to about 60 wt % of the total fiber weight.

According to certain embodiments of the invention comprising multi-component fiber and/or filament structures, the antimicrobial formulation may be added to any or all parts of the fiber and, preferably will be added in at least a phase in contact with the outside surface of the fiber, an example being a fiber with the antimicrobial disposed toward the outside surface of fiber in the sheath of a sheath/core bicomponent fiber.

The nonwoven of the invention may also comprise fine fibers containing the antimicrobial formulation where fine fibers include any fibers having an average fiber diameter that is less than about 8 microns and is produced from a molten polymer formulation. Nonlimiting examples of fine fibers may include a meltblown fiber, a melt fibrillated fiber, a rotational spun fiber, and an electrospun fiber comprising a polymer in combination with an antimicrobial heat labile component and carrier combination of the invention.

The nonwovens of the invention may be wetlaid, airlaid, carded or spun directly into a web. They may also be composites made from different layers of fibers, and those layers can be produced using different methods. Examples included but are not limited to a barrier fabric that combine layers of meltblown (M) and spunlaid continuous filaments (5) like the SMS, SMMS, or SSMMS constructs.

The nonwovens of the invention may, in addition to comprising fibers having a thermoplastic polymer containing the heat labile antimicrobial composition, contain other fibers having a thermoplastic polymer or other substance. Examples of these additional fibers comprising some other substance include but are not limited to, cotton, lyocell, viscose, rayon fibers as well as wood fibers.

The nonwovens of the invention can be stabilized by many different methods including but not limited to thermal bonding by calendering or hot air or steam or using ultrasonic energy, mechanical entanglement done by needling or hydroentanglement, or chemical bonding using a binder or a solvent and pressure.

### Test Methods

The basis weight of a nonwoven were measured using a procedure consistent with either the ASTM D756 or EDANA ERT-40,3-90 test methods. Measurement results were provided in units of mass per unit area in g/m² (gsm) and were obtained by weighing ten 10 cm by 10 cm samples of each of the samples followed by dividing by 0.01 m².

Air permeability was measured using a TexTest FX3300 Air Permeability Tester manufactured by TexTest AG, Zurich, Switzerland. The tester was used accordingly to the manufacturer instructions. The readings were obtained on a single ply of the nonwoven at a time using a 38 mm orifice and a pressure drop of 125 Pa, using a methodology that is consistent with the test method described in ASTM D-737. The readings were recorded as cubic meter per square meter per minute (m³/m²/min). The result reported for each Sample was the average of 10 readings.

The hydrohead resistance was measured using a Textest FX3000 Hydrostatic Head Tester manufactured by TexTest AG, Zurich, Switzerland. The tester was used in a way consistent with test method WSP 80.6 (05), with the exception that the rate of pressure increase was about 20 mBar per minute or about 20.4 cm of water per minute. Ten readings were taken for each sample and the average results were reported as the pressure in centimeter of water (cm of H₂O).

Measuring the average fiber diameter of continuous round fibers formed into a spunbond fabric is a common test for persons having ordinary skill in the art. The diameter of spun fibers typically ranges from about 10 to about 50 microns. The measurement typically involves measuring the width of the fibers using an optical microscope or a scanning electron microscope. For a round fiber, the measured width is equal to the diameter. The measurement device is first calibrated using an acceptable standard (e.g. an optical grid calibration slide 03A00429 S 16 Stage Mic 1MM/0.01 DIV from Pyser-SGI Limited, Kent, UK or SEM Target grid SEM NIST SRM 4846 #59-27F). A common method to select fibers at random is to measure the width of fibers along a line drawn between two points set across the piece being examined. This approach minimizes multiple measurements of the same fiber. Typically, the average diameter of the fibers is determined using a minimum of 10 fibers, and preferably 15 fibers, measured for a given layer in a sample. The average is calculated based on the total number of the fibers. Average diameter results are reported as micrometer (micron).

The average diameter may be used to calculate a theoretical surface area for 1 gram of the fiber. This calculation requires determining the perimeter of the fiber, the cross sectional area of the fiber, and the density of the fiber. The density of the fiber may be estimated from the density of its components at room temperature or measured using a method known in the art (e.g. gradient density column). For round filaments, a measure of the fiber diameter can be used to calculate the area and perimeter of its cross section.

For non-round filaments, the dimensions of the fibers may be obtained by first cross cutting the fibers and examining their cross sections under a microscope; recording the relevant dimensions needed for calculating the surface area of fiber for a given length (e.g. for a rectangular fiber those may be the thickness and width of the fiber, while for a trilobal or other complex shape fiber a meaningful measurement may be to measure the outside perimeter of the fiber cross section). Image analysis software may also be used in determining the fiber measurements needed to calculate surface area. For a fiber comprising polyolefin polymer, freezing the fibers before cutting them helps to obtain a better defined cross section of the fibers. The fiber dimensions may be calculated according to the following:

$$L = \frac{( 1 \text{/} D )}{C} \qquad ( 1 )$$

Where L is the length of 1 gram of fiber in cm, D is the density of the fiber in g/cm³, and C is the cross section of the fiber in cm².

CS=(2×C)+(P×L) (2)

Where CS is the calculated surface area per gram of fiber in cm², and P is the perimeter of the fiber cross section in cm.

The procedure used for determining the surface area of meltblown fibers having diameters less than 8 microns and formed into a nonwoven is similar to the procedure explained above for round continuous filaments with the exception that a scanning electron microscope is used to achieve a desired magnification. It is generally accepted that meltblown fibers have a substantially round cross section, therefore measurement of their width is consider the same as measuring their diameter. Because meltblown is a more variable process, there is a distribution of fibers having somewhat different diameters. Thus, the theoretical outside surface area of the fiber (CS) is calculated using the average diameter of the fibers.

The antimicrobial properties of the nonwoven samples were tested according to the “Antibacterial Finishes on Textile Materials: Assessment of” test method known as AATCC 100 with the following conditions: A) the method was performed using the Methicillin Resistant Staphylococcus Aurus (MRSA, ATCC 33592); B) a 0.01% solution of Triton X-100 was added to the inoculum to allow wetting of the sample because these are naturally hydrophobic; C) two carriers were tested per sample; D) exposure was at 20.0° C.; E) the neutralizer solution was Letheen Broth with 0.07% Lecithin and 0.5% Tween 80; F) the Agar plate medium was Tryptic soy agar with 5% sheep's blood; and G) a carrier was two pieces stacked together that were about 3.5 cm×7 cm.

The pore size distributions of the comparative examples and examples provided herein were measured using a capillary flow parameter. The instrument used for this measurement was a PMI Capillary Flow Porometer model CFP-1200-ACL-E-X-DR-2S, available from Porous Materials, Inc. of Ithaca, N.Y. A wetting fluid was used in the instrument having a surface tension of 15.9 mN/m, available under the trademark GALWICK® from Porous Materials, Inc.

The method used to measure the cumulative flow and pore size distribution was provided by the equipment manufacturer and is identified as a “Capillary Flow Porometry Test” using the “Wet up/Dry up” mode. A wrinkle free, clean circular sample is obtained from the Comparative Examples and Examples having a diameter of about 1.0 cm. The sample was saturated with the wetting fluid and then mounted into the cell of the PMI Capillary Flow Porometer, as per the manufacturer's instruction. When the mounting was complete, the apparatus was run by the apparatus software in the “Wet up/Dry up” mode to first record a flow vs. pressure curve for the sample saturated with the wetting fluid. When the flow v. pressure curve is recorded for the saturated sample, and the fluid has been expulsed from the pores, a flow vs. pressure curve was measured a second time on the same sample mounted in the instrument. The data generated includes the mean flow pore (“MFP”) where the pore size was calculated from the pressure where the half-dry curve intersects with the wet curve. The mean flow pore diameter was such that 50% of the flow is through pores larger than the mean flow pore. The measurement of pore size at 10% cumulative filter flow and the pore size at 25% cumulative filter flow can also be used as a way to characterize the presence of large pores.

### Example 1

The sample spunbond filaments of Example 1 were produced on a 0.5 meter wide pilot line. The line used had two extruders; each capable of being fed by a dry blend comprising polymer and an additive in the form of masterbatch. Each of the extruders were used to melt and mix the polymer composition fed to them and, they each fed a respective gear pump that controlled the flow of the polymer/masterbatch composition being fed to a die equipped with distribution plates and a spinneret producing sheath/core bicomponent continuous filaments. On the pilot line, the filaments were extruded from the spinneret and stretched while in the molten state by the force applied using a pneumatically driven slot attenuator. Quench air was blown on the bundle of filaments in the space between the spinneret and the attenuator in order to solidify the surface of the filaments. As the filaments exited the attenuator, they were blown toward and deposited on a moving belt to form a web with substantially random fiber orientation. The web formed on the moving belt was then consolidated by calendering using an embossed and a smooth heated roll. The formulation of the sheath and the core for some of the samples was the same, while the formulation of the sheath and the core of the remaining samples were different.

A spinneret having 1162 capillaries and a total throughput of about 0.5 gram per hole was used in the manufacture of the samples to achieve a targeted basis weight of about 38 gsm.

Some of the samples, as further described herein, were manufactured using INTRAGUARD™ SMT 1000 (“SMT 1000”) AND INTRAGUARD™ SMT 2000 (“SMT 2000”) supplied by Scientific Molecular Technologies, One Tower Lane, Suite 1700, Oakbrook Terrace, Ill. 60181 USA. SMT 1000 was a standard pellet extruded masterbatch comprising about 40 wt % of an antimicrobial heat labile component, as further described herein; about 10 wt % of a silica carrier; and about 50 wt % of a polypropylene. The antimicrobial heat labile component includes about 30 wt % of didecyldimethyl ammonium chloride, about 22 wt % of benzyl C-12-16 alkylidimethyl chlorides, about 17 wt % of benzathonium chloride, about 9 wt % of cetrimonium chloride, and about 22 wt % of N-(3-aminopropyl)-N-dodecylpropane-1,3 diamine.

SMT 2000 was a standard pellet extruded masterbatch comprising about 40 wt % of an antimicrobial heat labile component, as further described herein; about 10 wt % of a silica carrier; about 40 wt % of a polypropylene; and about 10 wt % of a high molecular weight silicone. The antimicrobial heat labile component for SMT 2000 is the same as that used for SMT 1000 and includes about 30 wt % of didecyldimethyl ammonium chloride, about 22 wt % of benzyl C-12-16 alkylidimethyl chlorides, about 17 wt % of benzathonium chloride, about 9 wt % of cetrimonium chloride, and about 22 wt % of N-(3-aminopropyl)-N-dodecylpropane-1,3 diamine.

### Sample 1

Sample 1 was a comparative sample where the same formulation consisting essentially of CP 360H, which is a narrow molecular weight 34 MFR polypropylene homopolymer supplied by Braskem America, 1735 Market Street, Philadelphia Pa., 19103 USA, was fed to each of the extruders producing the sheath and core having a ratio by weight of 1:1 of the bicomponent filamanet of Sample 1.

### Sample 2

The formulation for the core of the filament of Sample 2 consisted of 85 wt % of CP360H and 15 wt % of SMT 1000, while the formulation for the sheath consisted of 85 wt % of CP360H and 15 wt % of SMT 2000. The ratio by weight of the sheath to core of the filament of Sample 2 was 1:1.

### Sample 3

The formulation for the core of the filament of Sample 3 consisted of 75 wt % of CP360H and 25 wt % of SMT 1000, while the formulation for the sheath consisted of 75 wt % of CP360H and 25 wt % of SMT 2000. The ratio by weight of the sheath to core of the filament of Sample 3 was 1:1.

### Sample 4

The formulation for the core of the filament of Sample 4 consisted essentially of CP360H, while the formulation for the sheath consisted of 85 wt % of CP360H and 15 wt % of SMT 2000. The ratio by weight of the sheath to core of the filament of Sample 4 was 1:4. Process temperatures for each of the polymer streams fed to the die were adjusted to maintain good spinnability and to minimize heat exposure for the formulation fed to the sheath of the filament.

### Sample 5

The formulation for the core of the filament of Sample 5 consisted essentially of CP360H, while the formulation for the sheath consisted of 50 wt % of CP360H and 50 wt % of SMT 2000. The ratio by weight of the sheath to core of the filament of Sample 4 was 1:4. Process temperatures for each of the polymer streams fed to the die were adjusted to maintain good spinnability and to minimize heat exposure for the formulation fed to the sheath of the filament.

Tables 1-A and 1-B provide the process conditions for each of Samples 1-5, while Tables 2-A and 2-B identify the test results for the nonwovens produced from the bicomponent filaments of each of Samples 1-5. As used herein, the language “nonwoven of Sample” means the nonwoven manufactured from the filament of the numbered sample according to the method provided herein.

TABLE 1-A
Process Condition for the Production of Spunbond Fibers of Samples 1-3

| | | Sample 1 | | Sample 2 | | Sample 3 | |
|---|---|---|---|---|---|---|---|
| Process condition | Units | Core | Sheath | Core | Sheath | Core | Sheath |
| Throughput per | ghm | 0.25 | 0.25 | 0.25 | 0.25 | 0.25 | 0.25 |
| capillary | | | | | | | |
| Extruder zone 5 | ° C. | 235 | 236 | 195 | 198 | 199 | 199 |
| temperature. | | | | | | | |
| Extruder zone 6 | ° C. | 239 | n.a. | 197 | n.a. | 199 | n.a. |
| temperature | | | | | | | |
| Gear pump outlet | ° C. | 238 | 243 | 228 | 229 | 208 | 208 |
| temperature | | | | | | | |
| Pump outlet pressure | kPa | 2233 | 3137 | 2640 | 4054 | 2978 | 4847 |
| Extruder RPM | RPM | 15 | 20.6 | 15 | 25.6 | 31.6 | 33.7 |

TABLE 1-B
rocess Conditions for the Production of Spunbond Fibers of Samples 4-5

| | |
|---|---|
| Sample 4 | Sample 5 |

| | Units | Core | Sheath | Core | Sheath |
|---|---|---|---|---|---|
| Throughput per | ghm | 0.40 | 0.10 | 0.40 | 0.09 |
| capillary | | | | | |
| Extruder zone 5 | ° C. | 221 | 164 | 221 | 182 |
| temperature. | | | | | |
| Extruder zone 6 | ° C. | 218 | n.a. | 221 | n.a. |
| temperature | | | | | |
| Gear pump outlet | ° C. | 202 | 197 | 210 | 199 |
| temperature | | | | | |
| Pump outlet pressure | kPal | 3661 | 3068 | 3503 | 2799 |
| Extruder RPM | RPM | 22.4 | 18.7 | 22.4 | 17.6 |

TABLE 2-A
Test Results for Nonwovens Manufactured
from Filaments of Samples 1-3

| Test method | Units | Sample 1 | Sample 2 | Sample 3 |
|---|---|---|---|---|
| Basis weight | gsm | 39.3 | 36.9 | 35.2 |
| Air permeability | m³/m²/min | 92 | 105 | 137 |
| Hydrohead | cm of H₂O | 16 | 16 | 11 |
| resistance | | | | |
| Fiber diameter | micron | 18 | 17.9 | 21.3 |
| Calculated | cm²/g | 2469 | 2483 | 2087 |
| surface per | | | | |
| gram of fiber¹ | | | | |
| AATCC 100 | % (log₁₀) | n.a. | No | 33.8 (0.18) |
| bacterial | | | Reduction | |
| reduction after 3 | | | | |
| minutes | | | | |
| AATCC 100 | % (log₁₀) | No | n.a. | >98.1 (>1.73) |
| bacterial | | Reduction | | |
| reduction after 30 | | | | |
| minutes | | | | |

TABLE 2-B
Test Results for Nonwovens Manufactured
from Filaments of Samples 4-5

| Test method | Units | Sample 4 | Sample 5 |
|---|---|---|---|
| Basis weight | gsm | 39.3 | 38.8 |
| Air permeability | m³/m²/min | 99 | 99 |
| Hydrohead | cm of H₂O | 15 | 7 |
| resistance | | | |
| Fiber diameter | micron | 18 | 19.2 |
| Calculated | cm²/g | 2469 | 2315 |
| surface per | | | |
| gram of fiber¹ | | | |
| AATCC 100 | % (log₁₀) | 20.4 (0.10) | >98.1 (>1.73) |
| bacterial | | | |
| reduction after 3 | | | |
| minutes | | | |
| AATCC 100 | % (log₁₀) | n.a. | >98.1 (>1.73) |
| bacterial | | | |
| reduction after 30 | | | |
| minutes | | | |

¹The density of the fiber was not measured but was estimated at about 0.9 gram/cm3 since the predominant component is homopolymer polypropylene.

As a person of ordinary skill in the art would understand, the concentration of the antimicrobial component in the sheath may be calculated by:

% AMₛₕₑₐₜₕ=% AM_{MB}%MBₛₕₑₐₜₕ (3)

Where AM represents antimicrobial and MB represents masterbatch.

Additionally, the concentration of antimicrobial in the sheath relative to the total weight of the fiber may be found by:

$$\% {AM}_{sheath}^{fiber} = \% {AM}_{sheath} \frac{{Ratio}_{sheath}}{{Ratio}_{sheath} + {Ratio}_{core}} \qquad ( 4 )$$

Where the ratio is the ratio by weight of the sheath and the core as subscripted.

Using these equations, the information in Table 3 can be generated.

TABLE 3
Calculated Concentration of Antimicrobial in the Sheath

| | Sam- | Sam- | Sam- | Sam- | Sam- |
|---|---|---|---|---|---|
| | ple 1 | ple 2 | ple 3 | ple 4 | ple 5 |
| Sheath:Core Weight Ratio | 1:1 | 1:1 | 1:1 | 1:4 | 1:4 |
| % by weight Masterbatch in | 0% | 15% | 25% | 15% | 50% |
| Sheath | | | | | |
| % by weight Masterbatch in | 0% | 15% | 25% | 0% | 0% |
| Core | | | | | |
| % by weight Masterbatch in | 0% | 7.5% | 12.5% | 3% | 10% |
| Sheath Relative to Total | | | | | |
| Weight of Fiber | | | | | |
| % by weight Antimicrobial in | 0% | 6% | 10% | 6% | 20% |
| Sheath (40 wt % Antimicrobial | | | | | |
| in Masterbatch) | | | | | |
| % by weight Antimicrobial in | 0% | 3% | 5% | 1.2% | 4% |
| Sheath Relative to Total | | | | | |
| Weight of Fiber | | | | | |

Samples 1, 2 and 4 are provided as comparative examples while Samples 3 and 5 are representative of exemplary embodiments of the invention.

The nonwoven of Sample 3 showed a high efficacy at killing the MRSA bacteria as shown by the results obtained for the AATCC 100 test after 30 minutes exposure, while the nonwoven of Sample 5 showed a very high kill rate observed after 3 and 30 minutes of exposure. The results for the nonwoven of Sample 5 have a kill rate for MSRA that has never been observed before for a nonwoven comprising conventional melt dispersed antimicrobial agent. While not intending to be bound by theory, the performance of the nonwoven of Sample 5 appears to be due to the combined effect of: A) a high surface to weight ratio for the filaments exposed on the surface of this nonwoven and comprising the antimicrobial formulation; B) the low melt processing temperature used for the sheath formulation; and C) the high concentration of the antimicrobial agent at the surface of the fiber.

The results from the nonwoven of Sample 5 when compared to the results for the nonwovens manufactured form the other Samples suggest that it is not the total amount of antimicrobial agent or agents available in the fiber that is critical; rather, the results show that the concentration of the antimicrobial near the surface of the fiber is more critical. This suggests that a bicomponent fiber with a sheath having a relatively high concentration of the heat labile antimicrobial used for this invention could deliver better results than for a nonwoven made of fiber where the total amount of the same heat labile antimicrobial is greater while the concentration near the surface is less.

The impact of using as low processing temperature is further illustrated by comparison of the results for the nonwoven of Sample 2 and the nonwoven of Sample 4, The nonwoven of Sample 4 had the same antimicrobial formulation in its sheath as the nonwoven of Sample 2; however, the filament of Sample 4 was exposed to a lower melt temperature than the filament of Sample 2. The result was a noticeable difference in bacteria reduction after only 3 minutes of exposure for the nonwoven of Sample 4 versus the nonwoven of Sample 2.

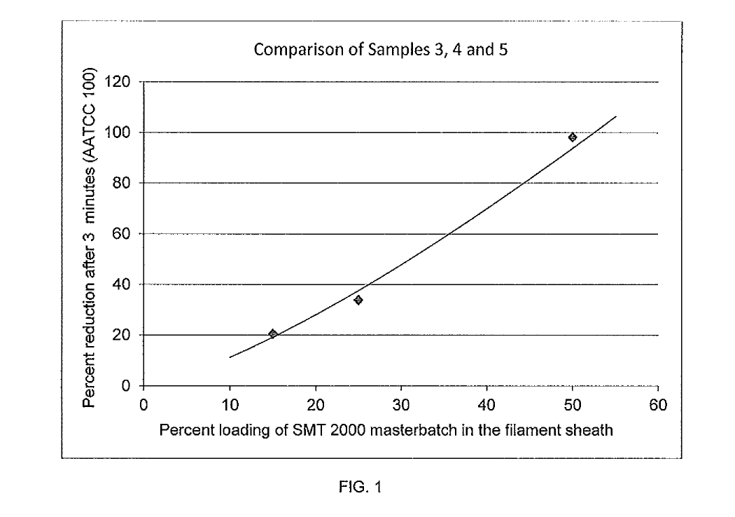

The impact of concentration at the surface of the filaments is illustrate in FIG. 1 where percent reduction in bacteria is compared to SMT 2000 masterbatch loading in the sheath for the nonwovens of Sample 3, 4 and 5, the filaments of each of which also had their sheath produced using lower processing temperatures.

### Example 2

The Sample of meltblown of Example 2 were produced on a Reicofill 1.1 meter wide pilot line. All of the samples were produced at a throughput of about 53 kilograms per hour or about 48 kg/h/m. The die tip 35 capillaries or holes per inch.

### Sample 6

The meltblown line was fed MF650X, which is a 1200 MFR meltblown polypropylene polymer manufactured from a metallocene catalyst by Equistar Chemicals, LP, LyondellBasell Tower, Suite 300, 1221 McKinney St., Houston, Tex. 77010 USA. The meltblown sample was manufactured at a target basis weight of 15 gsm.

### Sample 7

Sample 7 was made using the same formulation of Sample 6 except that the process conditions were modified to produce a target basis weight of the meltblown of 38 gsm.

### Sample 8

Sample 8 was made from a blend of 85 wt % of MF650X and 15 wt % of SMT 100. The target basis weight of the meltblown was 15 gsm.

### Sample 9

Sample 9 was made from the same formulation of Sample 8 except that the target basis weight of the meltblown was 38 gsm.

### Sample 10

Sample 10 was made from a blend of 75 wt % of MF650X and 25 wt % of SMT 100. The target basis weight of the meltblown was 15 gsm.

### Sample 11

Sample 11 was made from the same formulation of Sample 10 except that the target basis weight of the meltblown was 38 gsm.

Key process conditions for the manufacture of the meltblown of Samples 6-11 are included in Table 4, while some test results for these samples are included in Table 5.

TABLE 4
Key Process Conditions for the Production of Meltblown of Samples 6-11

| | | Sample | | | | | |
|---|---|---|---|---|---|---|---|
| Process conditions | Units | 6 | 7 | 8 | 9 | 10 | 11 |
| Throughput | kg/h | 53 | 53 | 53 | 53 | 53 | 53 |
| Melt temperature in | ° C. | 267 | 267 | 238 | 238 | 238 | 237 |
| the die | | | | | | | |
| Die Pressure | Bar | 5 | 5 | 8 | 8 | 11 | 11 |
| Primary air | ° C. | 260 | 260 | 240 | 240 | 240 | 237 |
| temperature | | | | | | | |
| Primary air volume | m³/h | 1100 | 900 | 1300 | 1300 | 1300 | 1300 |
| Secondary air | ° C. | 15 | 15 | 15 | 15 | 15 | 15 |
| temperature | | | | | | | |
| Distance of die to | mm | 150 | 150 | 150 | 150 | 150 | 150 |
| collector (DCD) | | | | | | | |
| Belt speed | m/min | 53.3 | 21.0 | 53.3 | 21.0 | 53.3 | 21.0 |

TABLE 5
Test Results for Meltblown of Samples 6-11

| | | Sample | | | | | |
|---|---|---|---|---|---|---|---|
| Test results | Units | 6 | 7 | 8 | 9 | 10 | 11 |
| Basis Weight | gsm | 15.3 | 38.7 | 19.5 | 38.1 | 14.7 | 38.3 |
| Air permeability | m³/m²/min | 19 | 7.3 | 76 | 15 | 139 | 33.5 |
| Average fiber | micron | 1.9 | 1.9 | 3.6 | 3.7 | 3.1 | 2.9 |
| diameter | | | | | | | |
| Mean Flow Pore | micron | 15 | 11.5 | 36 | 17 | 60 | 24 |
| Calculated surface | cm²/g | 23392 | 23392 | 12346 | 12012 | 14337 | 15325 |
| per gram of fiber¹ | | | | | | | |

¹ The density of the fiber was not measured but was estimated at about 0.9 gram/cm3 since the predominant component is homopolymer polypropylene.

Many modifications and other embodiments of the invention set forth herein will come to mind to one skilled in the art to which this invention pertains having the benefit of the teachings presented in the descriptions herein and the associated drawing. It will be appreciated by those skilled in the art that changes could be made to the embodiments described herein without departing from the broad invention concept thereof. Therefore, it is understood that this invention is not limited to the particular embodiments disclosed, but it is intended to cover modifications within the spirit and scope of the present invention as defined by the appended claims.

That which is claimed:

1. A fiber comprising:
a surface having a first concentration of an antimicrobial; and
a center having a second concentration of the antimicrobial, wherein the first concentration is greater than the second concentration.

2. The fiber according to claim 1, wherein the antimicrobial comprises an antimicrobial heat labile component and a carrier.

3. The fiber according to claim 1, wherein a surface area of the fiber is at least about 1070 cm²/g.

4. The fiber according to claim 1, wherein the first concentration is from about 3.5 wt % to about 12 wt % based upon the total weight of the fiber.

5. The fiber according to claim 4 having a kill rate of at least about 95% (log₁₀) after 30 minutes as measured by AATCC 100 test.

6. The fiber according to claim 4 having a kill rate of at least about 95% (log₁₀) after 3 minutes as measured by AATCC 100 test.

7. The fiber according to claim 4 having a kill rate of at least about 98% (log₁₀) after 3 minutes as measured by AATCC 100 test.

8. The fiber according to claim 2, wherein the second concentration is less than about 50% of the first concentration.

9. The fiber according to claim 1, wherein the fiber is a bicomponent fiber defined by a sheath and a core, and a concentration of the antimicrobial in the sheath is greater than a concentration of the antimicrobial in the core.

10. The fiber according to claim 9, wherein the concentration of the antimicrobial in the sheath is from about 3.5 wt % to about 12 wt % based upon the total weight of the fiber.

11. The fiber according to claim 10, wherein the concentration of the antimicrobial in the core is at most about 50% of the concentration of the antimicrobial in the sheath.

12. The fiber according to claim 1, wherein the concentration of the antimicrobial in the sheath is from about 9 wt % to about 25 wt %.

13. A nonwoven comprising a fiber, wherein a concentration of an antimicrobial at a surface of the fiber is greater than a concentration of the antimicrobial at a center of the fiber and the fiber having a surface area that is at least about 1070 cm²/g.

14. A method of manufacturing a bicomponent fiber comprising:
combining an antimicrobial with a first polymer; and
forming a sheath of the bicomponent fiber from the first polymer and a core of the bicomponent fiber from a second polymer.

15. The method of claim 14, additionally comprising combining the antimicrobial with the second polymer, wherein the concentration of the antimicrobial in the sheath is greater than the concentration of the antimicrobial in the core.

16. The method of claim 14, wherein the antimicrobial comprises an antimicrobial heat labile component and a carrier.

17. The method of claim 14, wherein a surface area of the sheath is at least about 1070 cm²/g.

18. The method of claim 17, wherein a concentration of the antimicrobial at a surface of the sheath is from about 3.5 wt % to about 12 wt % based upon the total weight of the bicomponent fiber.

19. The method according to claim 18, wherein the bicomponent fiber having a kill rate of at least about 95% (log₁₀) after 30 minutes as measured by AATCC 100 test.

20. The method according to claim 18, wherein the bicomponent fiber having a kill rate of at least about 95% (log₁₀) after 3 minutes as measured by AATCC 100 test.
